(19) United States
(12) Patent Application Publication
(10) Pub. No.: US 2022/0050105 A1
(43) Pub. Date: Feb. 17, 2022

(54) METHOD AND APPARATUS FOR DETECTING VIRUSES IN BIOLOGICAL SAMPLES

(71) Applicant: BOSTON MOLECULES, INC., Waltham, MA (US)

(72) Inventors: Taotao Lao, Newton, MA (US); Jiang Xu, Brookline, MA (US)

(21) Appl. No.: 17/096,076

(22) Filed: Nov. 12, 2020

Related U.S. Application Data
(60) Provisional application No. 63/064,911, filed on Aug. 12, 2020.

(51) Int. Cl. G01N 33/569; G01N 33/543; G01N 33/563; B01L 3/00

(52) U.S. Cl. CPC G01N 33/56983; G01N 33/54326; B01L 2300/0681; G01N 33/563; B01L 3/5027; G01N 33/54346

(57) ABSTRACT

The present invention relates to a process for detecting a virus that include the steps of: taking a biosample (e.g. saliva) suspected of containing a virus, mixing it with a solution comprising nanoparticles having easily detectable properties (e.g. a color) and also comprising contrasting microparticles (e.g. clear or white), each having attached chemical compounds (e.g. antibodies) that selectively bind to the virus to be detected (e.g. SARS-CoV-2). When suitably mixed together, virus present in the biosample may bind to the nanoparticles and to the microparticles, connecting the two. When the mixture is then passed through a microfluidic assembly with dimensions that trap the microparticles but pass unbound nanoparticles, the detection of the presence of nanoparticles bound to the microparticles at the microfluidic filter indicates the presence of the virus to be detected. The process may include a concentration step to accelerate binding the virus to the nano- and micro-particles.

### CROSS-REFERENCE TO RELATED APPLICATIONS

This application claims priority to U.S. Provisional Application No. 63/064911 filed on Aug. 12, 2020.

### FIELD OF THE INVENTION

The present invention relates to tests for rapid detection of the presence of viral material in a biological sample.

### BACKGROUND OF THE INVENTION

Rapid viral tests are qualitative or semi-quantitative in-vitro diagnostics (IVDs), used singly or in a small series, which typically involve non-automated procedures and have been designed to give a fast result. For the Severe Acute Respiratory Syndrome Coronavirus 2 (SARS-CoV-2), the virus responsible for the pandemic of Coronavirus Disease of 2019 (COVID-19), rapid tests such as antigen tests may take less than 15 minutes to produce a result, while other molecular tests may take as long as four hours, or even longer if samples need to be transported to a distant testing laboratory. By contrast, rapid viral tests may be used in clinical laboratories, near the point-of-care, or even in the home. They are relatively simple to perform, with results easy to interpret and therefore require limited training for test operators.

Examples of two types of SARS-CoV-2 rapid tests currently in use or in development are direct SARS-CoV-2 antigen detection and indirect antibody detection tests. Antigen detection tests detect viral components present during the infection in samples such as nasopharyngeal secretions.

Antigen tests have been slow to approval by any authorities for SARS-CoV-2 due to multiple reasons. Developing antibodies to capture an antigen is usually time consuming, and requires immunizing animals followed by monoclonal screening. Another reason is that the sensitivity is not as high when compared to Reverse Transcription Polymerase Chain Reaction (RT-PCR) RT-PCR, which involves exponential amplification.

There is therefore a need for a rapid viral test using monoclonal antibodies that can specifically capture and detect the SARS-CoV-2 virus by binding to viral protein.

### BRIEF SUMMARY OF THE INVENTION

The present invention relates to a rapid viral test using a pair of high affinity monoclonal antibodies that can specifically capture and detect the SARS-CoV-2 virus by binding to viral proteins. Combined with a filter implemented using microfluidic technology, we present a rapid diagnostic device that is designed to detect as low as 5 copies/μL of viral particles from saliva in only 5-15 minutes, with a sensitivity of approximately 90% and a specificity of approximately 90%. The test is suitable for use in clinical laboratories, near the point-of-care, or in the home. The simplicity of manufacturing the device lends itself to rapid scale-up and low cost to complement molecular tests, support decentralized testing capacity, and contribute to the pandemic control.

To execute the rapid viral test, a viral sample is taken (preferably a saliva sample) and mixed in a solution containing small, colored nanobeads as well as large, uncolored (e.g. clear or white) microbeads, with each type of bead having attaching to one or more antibodies for the virus under test.

If the virus under test is present in the mixture, the virus can connect to both the antibodies on each of the beads, and therefore form a molecular link between the small, colored nanobeads and the larger uncolored microbead, making a larger combined viral object comprising a large uncolored microbead with one or more viruses and colored nanobeads attached thereto.

When injected into a microfluidic array having an array spacing selected to correspond to the dimensions of the microbeads, the array functions as a filter, with the smaller, unattached colored nanobeads free to pass, but the larger, combined viral objects (which have colored nanobeads attached) becoming trapped in the array. The concentration of colored nanobeads forms an easily visible concentration of color that indicates the presence of virus.

Although the above description uses colored nanobeads as the marker to indicate the presence of the virus, nanobeads with any number of identifying characteristics could be used, including fluorescent nanobeads, luminescent nanobeads, magnetic nanobeads, or radioactive nanobeads.

### BRIEF DESCRIPTION OF THE DRAWINGS

FIG. 1 illustrates a flow chart of an embodiment of a process for detecting a virus according to the invention.

FIG. 2 illustrates schematically the steps of the process of FIG. 1

FIG. 3 illustrates a schematic view of a microfluidic channel assembly according to embodiments of the invention.

FIG. 4 illustrates a schematic top-view of a layout for a microfluidic channel assembly according to embodiments of the invention.

FIG. 5 illustrates two enlarged portion of top-view layouts of the microfluidic channel of FIG. 4.

FIG. 6 illustrates schematically the process steps of a modified process with an additional concentration step according to the invention.

FIG. 7 presents a photograph of two vials, one clear and the other (red) containing Nanoparticles, to be used in a kit to facilitate implementation of an embodiment of the invention.

FIG. 8 presents a photograph of a pair of syringes connected by a filter to be used to concentrate the nanoparticles in a kit to facilitate implementation of an embodiment of the invention.

FIG. 9 presents 3 photographs illustrating the concentration step of a modified process according to an embodiment of the invention, with (a) the beginning, (b) the middle, and (c) the end of the transfer process.

FIG. 10 presents a photograph of a syringe containing a solution of nanoparticles attached to the input port of a microfluidic assembly according to an embodiment of the invention.

FIG. 11 presents three photographs a microfluidic assembly according to an embodiment of the invention after (a) flushing a solution containing virus, (b) flushing with a different solution containing virus, and (c) flushing with a solution containing no virus.

### DETAILED DESCRIPTIONS OF EMBODIMENTS

### I. Virus Test Description.

The disclosed process for virus detection can be used use for both paraprofessional diagnostic applications and potentially as an in-home screening test.

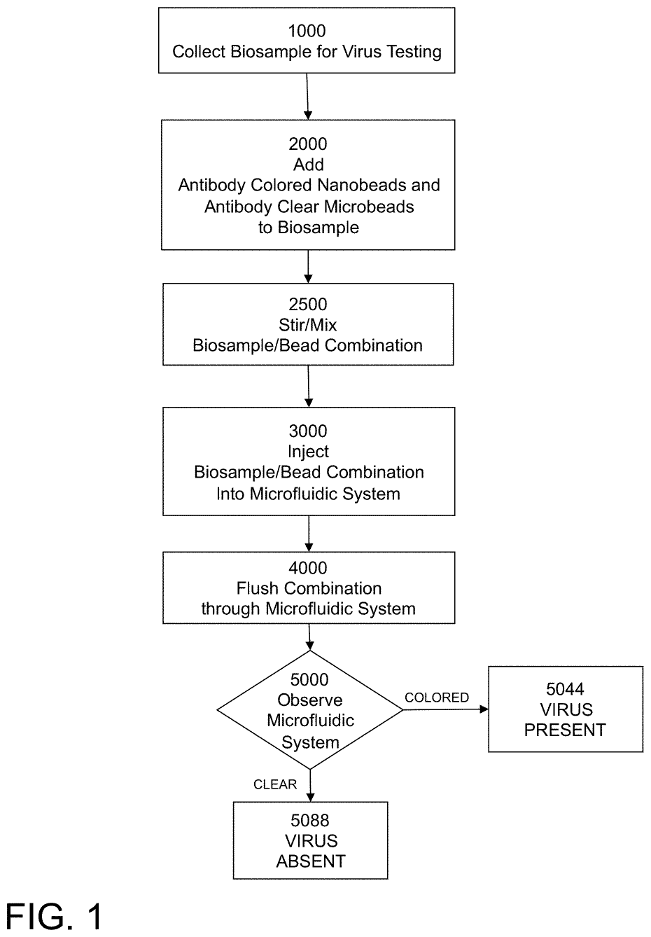

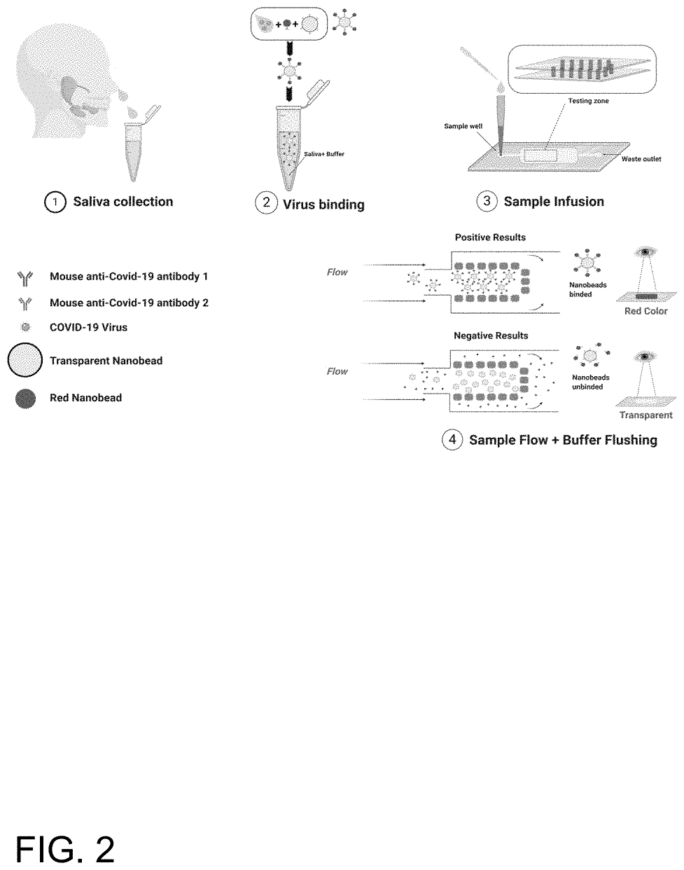

A flow chart and working mechanism of the method we have developed using a microfluidics diagnostic chip is briefly described below and illustrated in FIGS. 1 and 2. FIG. 1 presents a flow chart diagram of the steps in the process, while FIG. 2 presents illustrations of each step of the process.

Referring to FIG. 1, in the first step 1000, a biological sample (the biosample) to be tested for the presence of a predetermined virus (such as SARS-CoV-2) is collected into a sterile tube that also contains a buffer fluid. In one preferred embodiment, the biological sample can be a sample of the patient's saliva, collected by self-service.

Buffers for biological sample selection are commonly used in the art. In some embodiments of the invention, a phosphate-buffered saline (PBS) buffer with 1% bovine serum albumin (BSA) and 1% Triton® X-100 may be used, but other buffers at a pH level between 5 to 9 could be used as well. 1% Triton® X-100 is present to inactivate the virus and release proteins from viral particles. Use of a sterile plastic collection tube is preferred but not required, but care should be taken that no components that destroy viral protein function and structure (for example, guanidine) are present.

In the next step 2000, a solution comprising two types of beads is added to the biosample/buffer mixture. The solution will comprise both large clear (e.g. white or transparent) microbeads and small dyed/colored nanobeads. Commercially available pre-label protein A beads may be used to capture the viral antibodies, and treated with dimethyl pimelimidate (DMP) to crosslink the antibody to Protein A. This forms covalent bond that is not that easy to degrade.

The large clear microbeads may have a size between 1 and 40 microns in diameter, and have at least one type of antibody specific to antigens or proteins for the virus to be detected (such as BM-Ab-7 antibodies for SARS-CoV-2) attached to its surface.

The small dyed/colored nanobeads may have a size between 1 nanometer and 10 microns in diameter, but will typically have a diameter such as 340 nm or 410 nm. The nanobeads will also have at least one type of antibody also specific to antigens or proteins for the virus to be detected (such as BM-Ab-8 or BM-Ab-11 antibodies for SARS-CoV-2) attached to its surface. The antibodies on the small, colored nanobead will typically be a selected to be a different antibody from those used for the large, clear/transparent nanoparticle.

A listing of antibodies for SARS-CoV-2 that may be used in various embodiments of the invention are listed in the attached Appendix A.

In the next step 2500, the biosample/buffer/bead combination is stirred or mixed, to facilitate bonding between any virus and the antibodies on the beads.

In the next step 3000, the biosample /buffer/bead combination is injected to a planar microfluidic chip having a filtration/testing zone comprising a pillar-array. The pitch of the pillar-array is designed to be smaller than the diameter of large microbeads, but larger than the diameter of small nanobeads.

In the next step 4000, the biosample /buffer/bead combination is flushed through the microfluidic system.

In the next step 5000, the microfluidic system is observed by eye. If a virus with antigens suitable to both the antibodies on the nanobeads and the antibodies on the microbeads has bound the two beads into a larger, combined object, the combined objects will not pass through the filtration pillars, and instead aggregate within the testing zone. Because the combined object has colored nanobeads attached, the higher concentration of colored particles trapped in the filter zone will appear visibly as a colored concentration within the testing zone, allowing a rapid visible conclusion that virus is present 5044.

If, however, there is no virus present to bind the colored nanoparticles to the clear microparticles, the colored nanoparticles will all flush through the pillar array of the microfluidic system and the testing zone will remain clear, resulting in a negative test result and a conclusion that no virus is present 5088.

The presence of a concentration of color in the testing zone can therefore be interpreted as a positive indication of the presence of the virus to be detected (e.g. SARS-CoV-2), under the assumption that only the virus being sought would have antigens that pair with both of the antibodies in use on the nanobeads and the microbeads.

FIG. 2 presents an illustrated version of the steps listed in the flow chart of FIG. 1. The red color within the rectangle defined by the pillar array indicates the presence of the virus.

Although the illustration is shown using red colored nanobeads, beads having any visible color can be used. Fluorescent beads may also be used and examined under ultraviolet light if the additional contrast may allow more sensitive detection. Luminescent beads may be used, with the presence of a glowing line indicating the presence of a virus. Nanobeads with other detectable properties, such as magnetism or radioactivity, may also be used to provide a contrast to the microbeads.

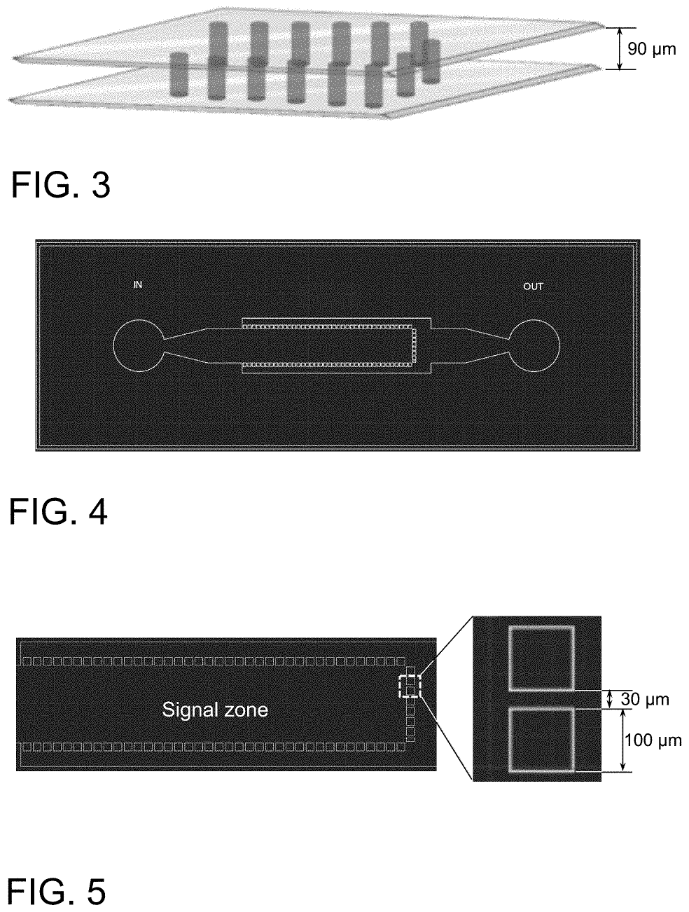

FIG. 3 illustrates a view from an angle of an embodiment of a microfluidic device that may be used in embodiments of the invention. A microfluidic assembly is manufactured from polydimethylsiloxane (PDMS), having a channel for fluid flow between two planar sheets, and with a Luer-lock aperture for fluid input on one side and an exit drain on the other.

The separation (channel height) between the two planar sheets may be on the order of tens of nanometers, and the exact dimensions will depend on the size of the microbeads in use. If 40 μm microbeads are used, a height of 70 to 90 μm between the planar sheets may be used. If 20 μm microbeads are used, the height may be 20 to 60 μm.

Between the two planar sheets are a number of pillars that form a pillar-array, with the gap between the pillars selected such that the microbeads cannot pass through the gaps in the pillar array, but the unattached colored nanobeads can pass through the array.

Fluid containing the biological sample along with the nano- and micro-beads is injected through the Luer-lock into the microfluidic channels, and forced through the pillar array. Clear/transparent microbeads are blocked from flowing, as are colored combinations of microbeads bound by virus to nanobeads, but unbound nanobeads are free to pass and are flushed through the system and out the out aperture, positioned on the end opposite the input aperture, and also on the side of the microfluidic device opposite the input hole.

In typical use, the microfluidic assembly will be positioned over a drain reservoir; fluid injected into the microfluidic channel from above flows through the device and then drains by gravity out and down through an aperture (the exit port) on the other end. In some embodiments, an additional drain tube may be attached to the exit port, and the drain reservoir may be contained using biohazard protocols, since active virus may still be present in the flushed fluid.

FIG. 4 illustrates a schematic top view of a layout of a microfluidic device suitable for use in some embodiments of the invention. The sample containing the biological sample and the micro- and nano-beads is injected from the left, and flows to the right through the pillar array. The spacing d between pillars is selected to be greater than the diameter of the nanobeads but smaller than the microbeads. When using 410 nm diameter nanoparticles with 40 μm microparticles, a gap d between the pillars no larger than 30 μm blocks the microparticles from passing but allows the nanoparticles to pass.

FIG. 5 shows a more detailed schematic top view of a portion of the microfluidic device of FIG. 4, with detailed dimensions shown in the inset.

### II. Prototype Example.

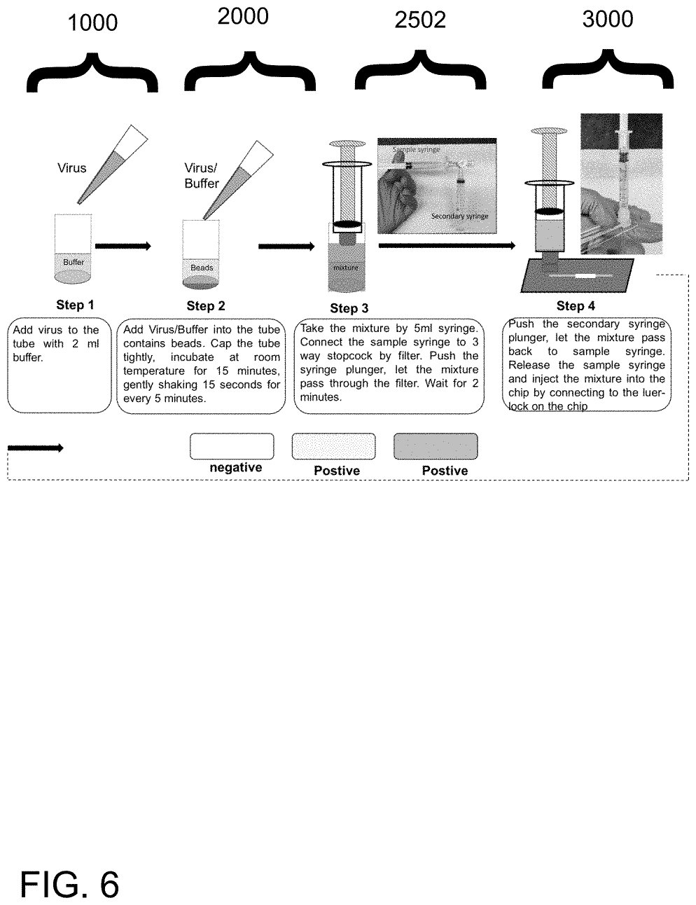

FIGS. 6-10 illustrate a prototype system that has been implemented as an embodiment of the invention.

In this embodiment, the mixing step 2500 is augmented by a filtration step prior to insertion into the microfluidic device. This augmented process serves to concentrate the components in the mixture, enhancing the formation of small to large bead bonding should a virus be present,

The augmented mixing process is illustrated in FIG. 6 In the augmented mixing step 2502, the biosample/bead mixture is drawn into a first syringe (˜5 mL) that is then in turn attached to a three-way Luer-lock connector. A second syringe is attached one of the other ports of the three-way Luer-lock connector. The connector additionally contains a 0.22 μm filter. The biosample/bead mixture is pressed using the first syringe through the filter into the second syringe. After a suitable amount of time (typically ˜2 minutes), the secondary syringe can be pressed to transfer the biosample/bead mixture back into the first syringe. The biosample/bead mixture so treated can then be transferred to the microfluidic device.

This accelerates the interaction and bonding between the nanobeads and microbeads. Without this step, after ˜15 minutes incubation with viral protein, lots of small beads are already bound with viral proteins, but the number of large beads is much less than small beads, and the large beads sink more easily to the bottom of the tube. There is therefore less opportunity for large beads to bind to a viral protein, especially when the total reaction volume is pretty large, for example, 1 mL, 2 mL or even 4 mL. These larger volumes require much longer incubation time to let limited number of large beads find small beads with viral proteins.

To detect small amount of viral protein in a limited time without any instrument is very difficult. One of the best benefits for the disclosed invention is that a large sample volume can be used. When many other viral detection technologies can only use a 10-200 μL sample, we can use 1 mL to 4 mL or even more without limitation. A device that can detect 5 copies/μL of virus is considered to be very sensitive device. This process can easily detect 5 copies/μL of virus, since we can enrich viral protein from 1 mL to 4 mL or even larger sample volume, which enhances the signal. The trade off in using the large volume is that you need more time to have small and large beads find each other.

Using a filter at this step 2502 forces the microbeads and nanobeads to concentrate at the filter, and therefore increases the probability of interaction for a microbead with a virally attached nanobead. Since the pore size of 0.22 μm in the filter is smaller than the size of the small nanobeads, both small and large beads will be exclusively squeezed in a very small volume next to the filter membrane. The concentration of large beads and small beads at this moment is very high, which gives a much better chance for large beads to recognize the viral-bound small beads, and allows viral binding of the large beads to the small beads. Without this step, it may take 30 minutes incubation time to get enough bonding to provide a good signal, while including this accelerated mixing step can reduce the time to results by 15 minutes or more.

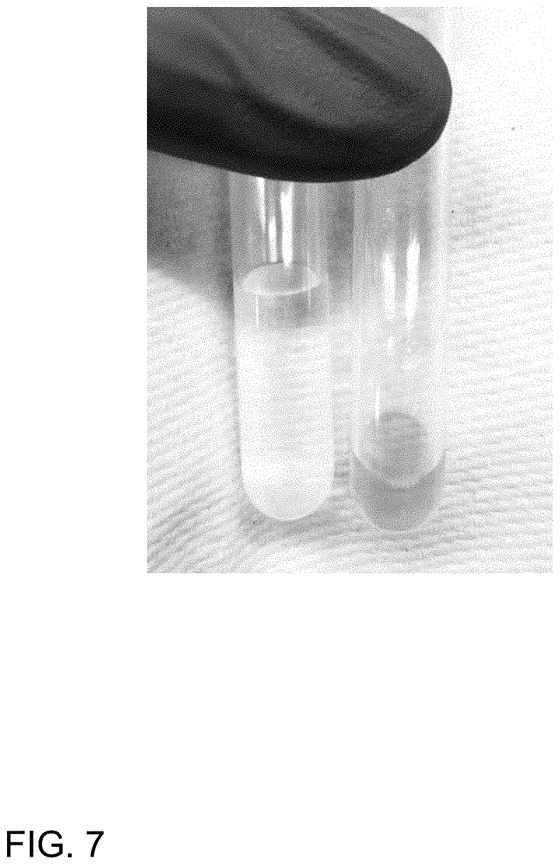

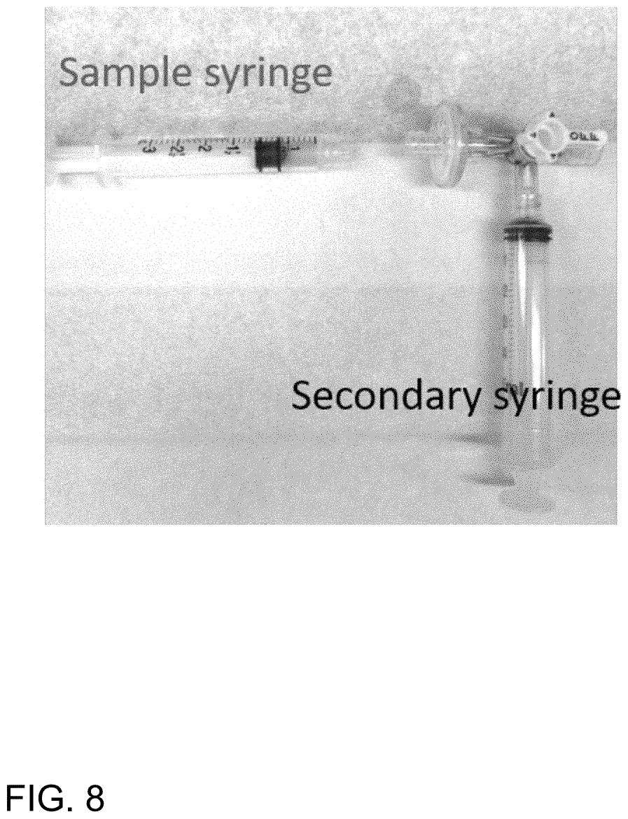

The materials for this rapid virus testing process can be packaged as a Virus Detection Test Kit. An example of the contents of such a kit are:
a. One (1) 5 mL plastic vial labeled as “Buffer”, containing 2 mL of buffer solution (clear solution) (illustrated in FIG. 7);
b. One (1) 5 mL plastic vial labeled as “Nanobeads”, containing a 0.5 mL solution with white microbeads and nanobeads suspended therein (red solution) (also illustrated in FIG. 7);
   the microbeads may be made from POROS™ MabCapture™ A Affinity Chromatography Resin, from Thermo Fisher Scientific, with for example, SARS-CoV-2 antibodies BM-Ab-7, while the dyed nanobeads may be, for example, NanoAct™ Cellulose Nanobeads (CNBs) from Asahi Kasei Fibers Corporation (Japan), provided with, for example, SARS-CoV-2 antibodies BM-Ab-8 and/or BM-Ab-11 attached;
c. One (1) PDMS microfluidic chip assembly manufactured from integrated with a Luer-lock connector on one side, and a drain port on the other side;
d. One (1) 2-foot long plastic tube for waste disposal from the drain port of the microfluidic chip;
e. One (1) waste collection bag;
f. Two (2) Luer-lock plastic syringes, e.g. those manufactured by BD (Becton Dickinson). One is called a first or sample syringe, another one is called secondary syringe, and is pre-fixed to a three-way Luer-lock connector (these are illustrated in FIG. 8);
g. One (1) Luer-lock 3-way connector;
h. One (1) 0.22 μm filter;
i. One (1) standard color chart of representative photos for interpreting results.

Additional materials that may be useful for executing embodiments of the method disclosed here but are not planned for inclusion a test kit, are:
a. Biosamples (e.g. saliva samples from a person to be tested);
b. SARS-CoV-2 virus samples;
c. Pipettes, pipette tips, dilution buffer, etc, for preparing virus dilutions;
d. Calibrated clock or timer;
e. A small test tube rack;
f. An absorbent underpad/paper (optional for waste collection);
g. Bleach for liquid waste disposal (optional).

The detailed processing steps for using the above described kit are as follows:

1. Preparation.
a. Place all materials on laboratory bench and allow equilibration to room temperature for 15 minutes prior to beginning.
b. While the test materials are equilibrating, prepare dilutions of the biosample (e.g. saliva) according to standard procedures.

2. Mix biosample (e.g. saliva) with buffer (corresponding to Step 1000)
a. Use lab micropipette to add 1 mL biosample (e.g. saliva) to the buffer solution. The total volume of saliva+buffer should be 3 mL.
b. Cap the vial tightly.
c. Gently swirl the solution for 30 seconds by hand to mix.

3. Combine biosample/buffer with nanobeads (corresponding to step 2000).
a. Remove the cap from the biosample/buffer solution vial, and use a micropipette to transfer contents to the vial that contains nanobeads. The total volume should be ˜3.5 mL, but given the loss of liquid during the operation, total volume of 3˜3.5 mL are also acceptable.
   Record the time of addition in the laboratory records.
b. Cap the vial containing the biosample/nanobead mixture,

4. Mix the biosample/nanobeads (corresponding to step 2500).
a. Shake the vial gently by hand for 30 seconds.
b. Place the vial in a small test tube rack and allow the biosample and nanobeads to incubate at room temperature for 15 minutes, gently inverting 5 times every 5 minutes.
c. Record the incubation period, including shaking times, in the laboratory records.

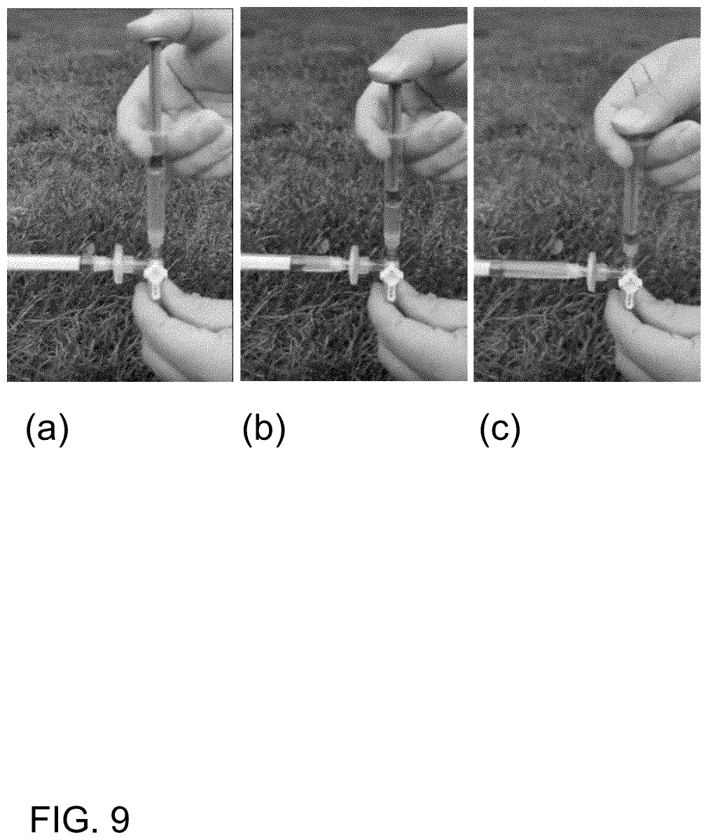

5. Modified Mixing Procedure (corresponding to step 2502), and illustrated in FIG. 9.
a. Draw all of mixed solution from the vial with the sample syringe.
b. Connect the sample syringe to the Luer end of the 0.22 μm filter.
   Double check the pre-connection of the filter and the secondary syringe to the three-way Luer-lock. Make sure all the connections are tight.
   Set the valve in the “OFF” position towards the outlet, or remaining, port on the three-way connector.
c. Use the connected syringes to mix the sample solution by gently pushing the sample through the filter, leaving 0.1˜0.2 mL residue in the sample syringe. This is illustrated in FIG. 9, with (a) in FIG. 9 showing the beginning of the process, with the colored biosample/nanobead mixture in the first or sample syringe, (b) in FIG. 9 showing the transfer from the first syringe to the second syringe in progress, and (c) in FIG. 9 showing the transfer to the second syringe completed.
   Do not use excessive force, or attempt to push sample through the syringe if there is resistance.
d. Allow incubation in the secondary syringe for two (2) minutes.
   Record the incubation starting and ending times in the laboratory records.
e. Gently push the solution from the secondary syringe back to the sample syringe. Do not use excessive force.
f. Remove the sample syringe from the three-way Luer-lock system.

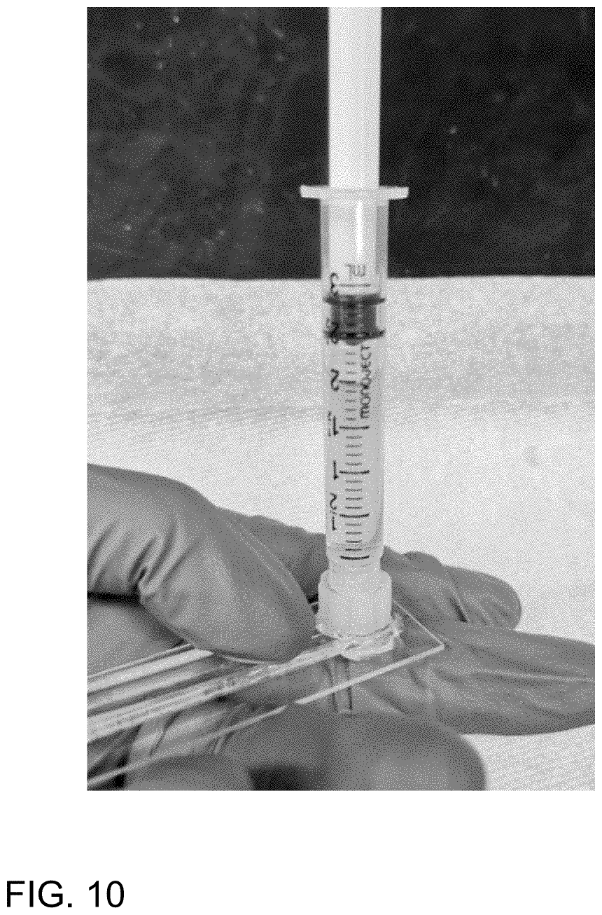

6. Injection into the Microfluidic Chip (corresponding to steps 3000 and 4000), and illustrated in FIG. 10.
a. Connect the sample syringe to the microfluidic chip tightly via the Luer-lock at the inlet. This is illustrated in FIG. 10.
b. Make sure the exit port of the microfluidic device is positioned to safely drain away from the device, either into a waste tube or a container/reservoir/bag that can collect biohazard waste.
   Insert the end of the waste tubing completely into the waste collection bag to prevent leaks or spills.
   The end of the waste tubing may also be placed directly into a bleach bucket, absorbent pad, or alternative means of disinfection and disposal per standard operating procedure.
c. Place the microfluidic chip on a firm and flat surface.
d. Check for the presence of bubbles in the sample. If there are bubbles in the bottom of syringe, gently flick the syringe with fingers to float the bubbles. Do not inject bubbles through the microfluidic chip.
e. Gently press plunger to inject the sample through the microfluidic chip.
   An optimal injection time is between 20 seconds to 1 minute.
   Do not inject the liquid faster than 10 seconds.
   Record the injection time in the laboratory records.

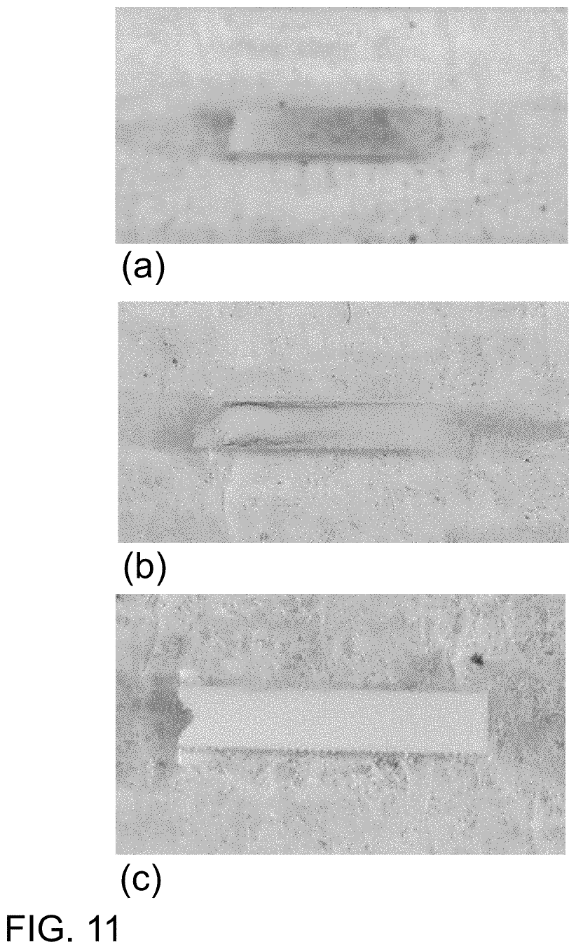

7. Observation of Results (corresponding to step 5000), and illustrated in FIG. 11.
a. Observe the microfluidic chip for color change.
   Compare the observation zone to the standard color chart.
   Note: Color change should be immediate.
b. Determine the test result according as POSITIVE (presence of color) as illustrated in both examples (a) and (b) of FIG. 11, or
   NEGATIVE (no color), as illustrated in example (c) of FIG. 11.
   Record the results in the laboratory records according to standard operating procedure.
c. Dispose of all used materials into a marked biohazard waste container.

### III. Additional Options.

Although several embodiments of the invention have been described, those skilled in the art may recognize that other options are possible.

In some embodiments, a sample from a nasal swab may be the biosample, instead of the saliva sample described in the examples above.

In some embodiments, a sample of blood, plasma, lymph, or another bodily fluid may be the biosample, instead of the saliva sample used in the examples above.

In some embodiments, although this has been developed with the detection of SARS-CoV-2 in mind (and may be especially useful when bonding to N-proteins of SARS-CoV-2), this process can be used for detecting other types of virus, small molecules, proteins, sugars, toxins, and anything else that can be recognized by antibody or receptors, and used to form bonds between nanoparticles and microparticles.

Although mixing using syringes and a 0.22 μm filter has been disclosed in the example presented above, other filter sizes and syringe configurations may be used for facilitating the binding of the virus to the colored nanobeads and to the clear microbeads.

Although colored nanobeads and uncolored microbeads of particular representative sizes have been disclosed, various sizes, shapes, and dimensions of nano- and micro-beads may be used according to the invention. The colored nanobeads may be colored using any number of dyes, hues, or even electromagnetic effects occurring on the surface or integral to the material of the nanoparticle, as long as there is a visible or otherwise easily detectable difference with the uncolored microbeads. Similarly, the uncolored microbeads may be white, black, grey, or transparent, as long as they can provide a contrast with the nanobeads that can be easily detected.

Likewise, the nano- and micro-particles need not be spherical beads, and nanoparticles and microparticles of varying shapes and sizes may be used, as long as the larger particles are uncolored (white or clear) and the nanoparticles are colored or otherwise detectable and distinguishable from the microparticles.

Although an example using a microfluidic assembly made from PDMS has been disclosed, other materials and techniques for microfluidic manufacturing, such as thermoset polyester (TPE) may also be used. Likewise, although the implementation in the embodiment described above uses passage through a microfluidics device to concentrate the combined viral objects, the pillar array is functioning similar to a filter, and as such, this function could be achieved using any number of structures that facilitate liquid flow that comprises a filter to trap the large viral objects while passing the unattached nanoparticles.

Additional information on various antibodies that may be used in embodiments of the invention may be found in the attached Appendix A, entitled “SARS-Cov-2 Antibody List” (1 page).

Additional information may be found in the attached Appendix B, entitled “Microfluidic device for detecting of components in samples” (8 pages).

Additional information on microfluidic assembly manufacturing techniques may be found in the attached Appendix C, entitled “Smart Materials Microfluidic channel fabrication” (6 pages).

With this Application, several embodiments of the invention, including the best mode contemplated by the inventors, have been disclosed. It will be recognized that, while specific embodiments may be presented, elements discussed in detail only for some embodiments may also be applied to others.

While specific materials, designs, configurations and fabrication steps have been set forth to describe this invention and the preferred embodiments, such descriptions are not intended to be limiting. Modifications and changes may be apparent to those skilled in the art, and it is intended that this invention be limited only by the scope of the appended claims.

What is claimed is:

1. A method for detecting viruses in biological samples, comprising the steps of:
obtaining a biological sample, said biological sample to be tested for the presence of a predetermined virus;
adding a combination of microparticles and colored nanoparticles to the biological sample, wherein
first antibodies corresponding to the predetermined virus have been attached to at least some of the colored nanoparticles, and
second antibodies corresponding to the predetermined virus have been attached to at least some of the microparticles;
mixing the biological sample with the nanoparticles and the microparticles, in such a manner that enables a virus present in the mixture to bind to both a colored nanoparticle and a microparticle;
filtering the mixture through a fluid channel assembly,
said assembly comprising a filtering structure that allows individual nanoparticles to pass while blocking the passage of the microparticles; and
observing the color of the filtering structure.

2. The method according to claim 1 wherein, the predetermined virus is SARS-CoV-2.

3. The method according to claim 1, wherein the color of the filtering structure includes red indicating presence of the predetermined virus or clear indicating absence of the predetermined virus

4. The method according to claim 1 wherein the step of obtaining a biological sample includes:
collecting a sample of human saliva and suspending in a buffer solution.

5. The method according to claim 1 wherein:
the colored nanoparticles are colored nanobeads, wherein said colored nanobeads include a diameter in the range of 1 nanometers to 10 micrometers, and
the microparticles are white microbeads, wherein said white microbeads include a diameter in the range of 1 micrometer to 400 micrometer.

6. The method according to claim 5 wherein:
the colored nanobeads include a diameter greater than or equal to 340 nanometers and less than or equal to 410 nanometers; and
the microparticles include a diameter in the range of 1 micrometer and 40 micrometer.

7. The method according to claim 6 wherein, the colored nanobeads include a dye on the surface of each nanobead.

8. The method according to claim 5 wherein, the colored nanobeads include dye incorporated into the matrix of the beads.

9. The method according to claim 4 wherein, the colored nanobeads are red.

10. The method according to claim 1 wherein,
the first antibodies are selected to recognize an epitope from an antigen corresponding to the predetermined virus, and the second antibodies are selected to recognize a different epitope from said antigen, such that the second antibodies are selected to be different from the first antibodies.

11. The method according to claim 2 wherein,
the first antibodies are selected from a group consisting of SARS-CoV 2 BM-Ab-8 and BM-Ab-11 antibodies; and
the second antibodies are SARS-CoV-2 BM-Ab-7 antibodies.

12. The method according to claim 1 wherein,
the fluid channel assembly comprises a microfluidic channel, having a height greater than the size of the majority of the microparticles; and wherein
the filtering structure comprises an array of pillars within the microfluidic channel.

13. The method according to claim 11 wherein,
the height of the microfluidic channel is in a range of 70 micrometers to 90 micrometers; and all gaps between the pillars in the array of pillars are less than 30 micrometers.

14. The method according to claim 1 wherein, the step of mixing the biological sample with the nanoparticles and the microparticles further includes the steps of:
placing a solution into a first syringe, where the solution includes the biosample, the nanoparticles, and the microparticles;
attaching the first syringe to an assembly, where the assembly includes a filter, and a second syringe;
ejecting the mixture from the first syringe through the filter, whereby said nanoparticles and said microparticles concentrate on the filter while fluid from the mixture passes into the second syringe;
waiting a predetermined amount of time; and
ejecting the fluid from the second syringe through the filter back into the first syringe.

15. The method according to claim 13, wherein:
the filter is selected to be a 0.22 μm filter.

16. The method according to claim 13, wherein:
the predetermined amount of time is between 1 and 3 minutes.

17. An method for detecting viruses in biological samples, comprising the steps of:
obtaining a biological sample, said biological sample to be tested for the presence of a predetermined virus;
adding a combination of microparticles and nanoparticles to the biological sample, wherein
first antibodies corresponding to the predetermined virus have been attached to at least some of the nanoparticles, and second antibodies corresponding to the predetermined virus have been attached to at least some of the microparticles;
mixing the biological sample with the nanoparticles and the microparticles, in such a manner that enables a virus present in the mixture to bind to both a nanoparticle and a microparticle;
filtering the mixture through a fluid channel assembly,
said assembly comprising a filtering structure that allows individual nanoparticles to pass while blocking the passage of the microparticles; and
observing the appearance of the filtering structure.

18. The method according to claim 17, wherein the nanobeads includes one of a group of nanobeads including fluorescent nanobeads, luminescent nanobeads, magnetic nanobeads and radioactive nanobeads.
